(19) United States
(12) Patent Application Publication
(10) Pub. No.: US 2009/0171239 A1
(43) Pub. Date: Jul. 2, 2009

(54) Sensory Test Device

(75) Inventors: Fredrik Juto, Stockholm (SE); Jan-Erik Juto, Stockholm (SE)

Correspondence Address: BIRCH STEWART KOLASCH & BIRCH, PO BOX 747, FALLS CHURCH, VA, 22040-0747, US

(73) Assignee: RHINOMED AB, STOCKHOLM (SE)

(21) Appl. No.: 12/084,402

(22) PCT Filed: Oct. 20, 2006
(86) PCT No.: PCT/SE2006/001191

§ 371 Date: Aug. 29, 2008

(30) Foreign Application Priority Data
Nov. 1, 2005 (SE) 0502412-0

(51) Int. Cl. A61B 19/00

(52) U.S. Cl. 600/557

(57) ABSTRACT

There is disclosed a device for testing tactile sensitivity of tissue, such as nasal mucosa, comprising an elongated portion to which there is attached a thread. The thread is adapted to be brought into contact with the tissue to be tested, wherein the thread is curved or otherwise bent so as to define a lateral contact point away from the axis of the elongated portion. Preferably, the thread is axially attached to the elongated portion, and exhibits a curvature or bent to define a lateral contact point about 10 mm or less away from the axis of the elongated portion.

### TECHNICAL FIELD

The present invention relates to devices for testing tactile sensitivity of tissue.

### TECHNICAL BACKGROUND

Instruments for testing tactile sensitivity are known in the prior art. For example, U.S. Pat. No. 3,662,744 discloses such instrument, for measuring cutaneous sensory perception. The instrument comprises an elongated tubular housing and a sleeve member extending axially from an end thereof. The instrument utilizes a monofilament for stimulating the skin of a patient by applying an axial force downwardly along the instrument.

Another example of a tactile sensory testing instrument is disclosed in U.S. Pat. No. 5,823,969. In this case, a monofilament test element is attached to a two-position pivot head, for switching between a protected position where the monofilament is housed inside a protective channel, and a test position where the monofilament extends vertically downwardly from the instrument handle.

The above-referenced prior art instruments are designed for testing the skin of a patient. However, the instruments can not be used for testing internal tissue, such as nasal mucosa, larynx or other mucosae in difficult to reach body cavities.

### SUMMARY

An object of the present invention is thus to provide a sensory measuring and/or testing device, which is suitable for testing tissue in difficult to reach locations.

This object is met by a device and a test kit as set forth in the appended claims.

Devices according to the present invention may be used for at least two types of tests. Firstly, the devices may be used for providing a measure of the tactile sensitivity of nasal mucosa, larynx or other mucosae in difficult to reach body cavities. While specifically designed to be utilized for such difficult to reach cavities, it will be appreciated that the device may also be used for more easily accessible parts of the body. Secondly, the devices may be used for measuring the topological distribution of tactile sensitivity across the surface of such mucosae, and as an aid for inferences about the underlying causes of observed distributions.

The inventive test device, or test stick, comprises an elongated portion, preferably in the form of a thin duct, for reaching the location to be tested, and a thread or filament extending from the elongated portion and reaching out, laterally with respect to the longitudinal dimension of the elongated portion, to contact the tissue to be tested. Optionally, the test device also comprises a handle for handheld operation, or an attachment for mounting in a mechanical manipulator.

Preferably, the test devices are designed as disposable devices adapted for single use.

In accordance with the present invention, there is proposed a test kit, comprising a plurality of test devices, wherein each test device within a kit is adapted to test for a different tactile sensitivity threshold by applying a different force to the tissue being investigated. Typically, a test kit will comprise between three and five individual test devices. By using these test devices according to a prescribed procedure, the operator, such as a medical doctor or a nurse, may classify the tactile sensitivity of a test subject as being normal or abnormal, as well as various degrees of sensitivity. Typically, an abnormal sensitivity may be classified as hypersensitive or hyposensitive. The operator may also make observations about the topology of the tactile sensitivity of the test subject across the tissue of interest, such as its nasal mucosa or other mucosae. Such observations may provide valuable input for diagnosis of various pathologies or for planning and scheduling surgical interventions.

During conduct of the test procedure, each test device is being used independently of the other devices in the same kit. Each device is used one at the time to effect a tactile stimulation of the tissue under investigation using a contact force dictated by each test device. For determining whether each stimulation has lead to a response, the subject may, for example, be asked to indicate whether its sensory threshold has been exceeded (e.g. using a push button or the like). By comparing the responses from the test subject to the force applied by each test device of the kit, the operator may determine or assess the tactile sensitivity of the subject.

Each test device in the kit is intended to be used using a sampling procedure, wherein measurements of tactile sensitivity from different locations of the tissue (e.g. nasal mucosa) are combined into an aggregate measure which is more representative of the overall tissue.

Preferably, each test kit has a specified procedure for the sequence in which each test device should be utilized to test tissue sensitivity of the subject. Such procedure may improve the reliability of measurements and allow the operator to classify the subject as having normal, hypersensitive or hyposensitive tissue.

Using a different procedure, the operator may assess the topological distribution of tactile sensitivity and draw inferences about underlying causes for the observed distribution.

### BRIEF DESCRIPTION OF THE DRAWINGS

In the following detailed description, reference will be made to the accompanying drawings, in which:

FIG. 1 schematically shows a device according to the present invention;

FIGS. 2-4 schematically show various ways of attaching the thread to the elongated portion of the test stick;

FIG. 5 schematically shows an alternative embodiment of the invention, wherein the force probability for the test stick is determined by a local weakening of the extending thread;

FIG. 6 schematically shows another alternative embodiment of the present invention, wherein the elongated portion is provided with a small angle at its free end;

FIG. 7 schematically illustrates how the inventive device is used for delivering a tactile stimulation to tissue;

FIG. 8 schematically shows a test kit, comprising a plurality of devices according to FIG. 1, according to the present invention;

FIG. 9 is a graph illustrating the force magnitude delivered by the inventive device;

FIG. 10 is yet another graph illustrating the force magnitude; and

FIG. 11 is a graph schematically illustrating the force magnitude delivered by respective devices of a test kit according to the present invention.

### DETAILED DESCRIPTION

Preferred embodiments according to the present invention will now be described, by way of example only, referring to devices having an elongated portion in the form of a duct, in which a thread is arranged and extending out from one end to provide the contact point for contacting tissue to be tested. As an example, reference will be made to testing of nasal mucosae, although it will be appreciated that similar devices and procedures may also be used for testing other kinds of tissue.

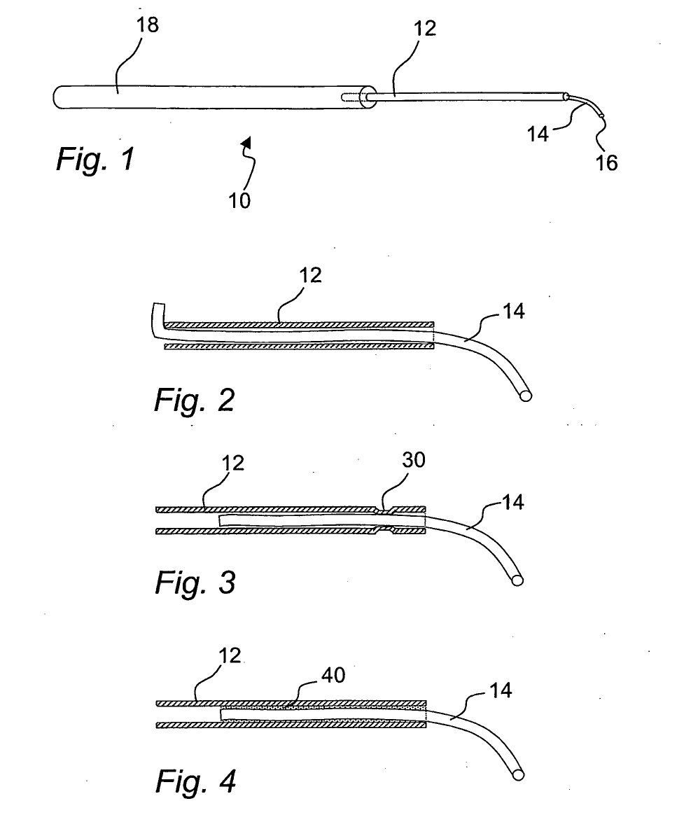

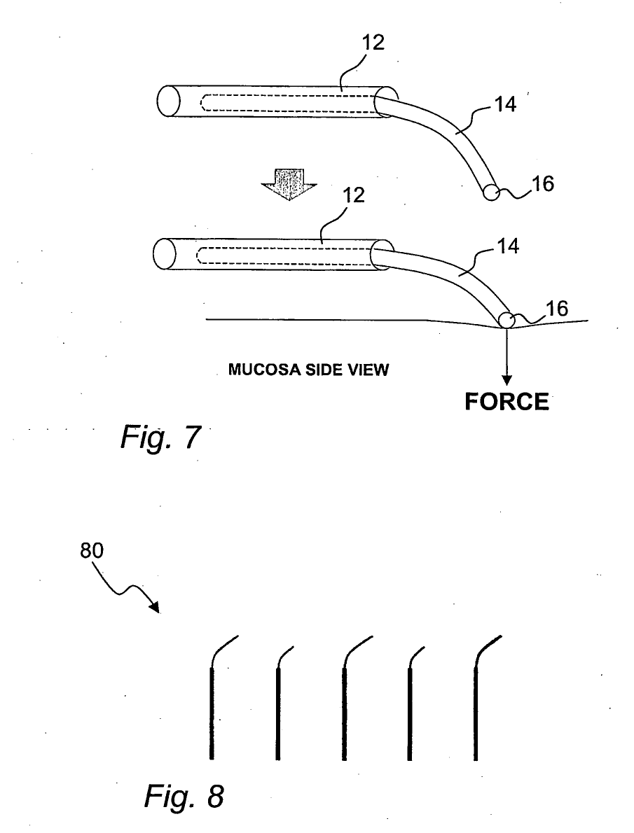

FIG. 1 schematically shows an embodiment of the present invention in the form of a test stick 10. The test stick comprises an optional handle 18, an elongated portion 12 in the form of a duct, and a thread 14 extending axially from the free end of the duct 12. Suitable attachment means is provided for attaching the elongated portion 12 to the optional handle 18. As shown in FIG. 1, the thread 14 intended for tactile stimulation of mucosa or similar tissue extends axially from the elongated portion 12. However, the thread is curved or otherwise bent in order to provide a lateral point of contact at the tip 16 of the thread. By lateral contact point, it is meant a contact point outside the axis of the elongated portion 12, i.e. off axis. However, the thread 14 need not, and will typically not, be designed to contact the tissue at normal angle at the tip 16. Rather, by the lateral movement of the test stick 10 during use, the thread 14 will be brought into contact with the tissue at an angle. By virtue of this design, the test stick 10 may be used for reaching into, for example, the nasal cavity of a person and to stimulate or test the nasal mucosa by the operator moving the stick sideways. During tactile stimulation using the inventive device, the elongated portion 12 (the duct) is kept more or less parallel to the mucosa, and the tip 16 of the thread 14 comes into contact with the mucosa due to its curvature or bend away from the longitudinal axis of the elongated portion 12. This is schematically illustrated in FIG. 7, showing how the tip of the thread is brought into contact with the mucosa. Typically, the lateral contact point 16 will be less than about 10 mm off the axis of the longitudinal portion. However, how far off-axis the contact point may be will be determined by the intended use and the size of the cavity in which the test stick is to be applied.

Keeping in mind that the devices according to the present invention should be able to operate in difficult to reach spaces, such as inside the nasal cavity of a subject, the test device 10 is to be applied sideways towards the mucosa in order to contact the mucosa with the tip 16 of the extending thread 14. The duct 12, or elongated portion, need not and should preferably not come into contact with the mucosa. By virtue of a suitable curvature or bend of the extending thread 14, contact with the mucosa is effected without bringing the duct 12 into contact at the same time.

For facilitating the operation, the operator will typically observe the tip 16 of the thread 14 as well as the mucosa through a microscope or similar during the test procedure, in order to ensure proper application of the test stick and in order to identify the locations on the mucosa at which the tactile stimulations is applied.

The thread 14 may be attached to the elongated portion 12 of the test stick 10 in any suitable manner. A few examples are given in FIGS. 2-4. FIG. 2 illustrates an embodiment in which the thread 14 is inserted through the duct 12 of the elongated portion and firmly attached in a frictional manner by means of a wedge or similar (illustrated by the sharp bend of the thread at the end of the elongated portion). This embodiment may prove advantageous from a manufacturing point of view, facilitating high volume production.

FIG. 3 shows another embodiment, in which the thread 14 is attached to the duct 12 by means of an indentation 30, providing a clamping fixture for the thread inside the duct. The indentation 30 may be located anywhere along the length of the duct 12, provided that the characteristics of the extending thread 14 remain. Further, the indentation 30 may be of any suitable length. This embodiment may prove advantageous if the elongated portion 12 of the test stick 10 is made from a deformable material.

Yet another embodiment is shown in FIG. 4, wherein the thread 14 is glued in place within the duct 12. The glue 40 may be applied anywhere along the length of the duct and fill any length of the duct, provided that the desired properties of the duct and the extending thread are obtained. This embodiment may have the advantage that a higher degree of repeatability is provided, leading to less variation in probable applied force for test sticks having the same rated stimulation force.

In all embodiments of FIGS. 2-4, the thread 14 has been given a suitable curvature or bend in order to fulfill the requirement that is should provide a lateral contact point although it extends substantially axially from the elongated portion 12 of the test stick. It is also possible, instead of using a smoothly curved thread, to employ a thread that has been given a localized bend for providing the lateral contact point of the test stick.

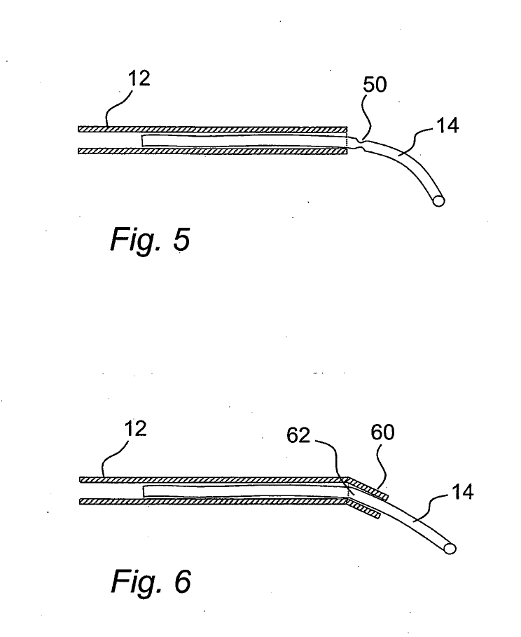

As explained elsewhere in this specification, the force probability of the inventive devices may be determined by the physical properties of the thread 14, such as by its diameter, stiffness, choice of material, curvature, uniformity or homogeneity. One example of providing the test stick with suitable force characteristics is by locally weakening the extending thread, as illustrated at reference 50 in FIG. 5, e.g. to a reduced diameter. FIG. 5, shows an embodiment of the inventive device for which the thread 14 has been locally weakened adjacent the free end of the elongated portion 12 (the duct). By making such local weakening asymmetrical about the circumference of the thread 14, it is possible to make provisions such that the thread will primarily bend in a single geometrical plane when subjected to a force. This may facilitate operation during use of the test stick.

Another embodiment is shown in FIG. 6, wherein the elongated portion 12 to which the extending thread 14 is attached has been provided with an angled free end 60 (or tip). In this case, the thread 14 may be curved or bent inside the duct, as illustrated at reference 62, rather than outside the duct. However, similar operation is provided.

It should be noted that the thread should have a comparatively small diameter, meaning that a point-like region, or only a very small region, is stimulated during the test procedure. By using the inventive test device, the operator may thus test whether the subject is able to sense the force applied by the test device through nerve endings located at this point or in its immediate proximity. In order to test the sensitivity of the mucosa or the density of nerve endings over a larger surface area, a sampling procedure must be employed in which the sensitivity is tested across several points (or small regions) of the mucosa.

For testing tactile sensitivity, the test stick should preferably be applied against 3-10 different points of the mucosa, the subject each time being asked whether it can sense the stimulation. The ratio of the number of positive measurements to the total number of applications of the test stick will give a measure, a “stick measure”, for this test stick. The stick measure is then compared to a threshold value in order to determine whether the overall result for this particular test stick is positive or negative. Preferably, in order to avoid false positive measurements during the procedure, the subject may be asked to respond several times also when the test stick is not in fact in contact with the mucosa. The magnitude of the tactile sensitivity of the mucosa follows a distribution determined by the topology of the nerve endings in the mucosa, as well as by other factors. For a test stick having pressure characteristics exactly matching the center of the mucosal sensitivity distribution, it may be expected that about half of the measurements would be negative and about half would be positive, giving a stick measure between 0 and 1. Hence, in order to have a result showing the sensitivity is above or below the pressure designated to the test stick used, a stick measure close to 0 or 1 should be obtained.

For mapping the topology of tactile sensitivity, or the topological distribution of nerve endings in the mucosa, the same test sticks are used as for the tactile sensitivity measurement. However, another test procedure is employed. Using a selected test stick, the operator will tactilely stimulate the mucosa at a plurality of points, typically more than 10 points, and for each of these points observe whether the subject has sensed the stimulation or not. The operator may chose to count the number of stimulations and the number of positive responses, or to make a subjective assessment of the sensitivity observed.

In general, when using the inventive devices, the operator is advised not to make the timing of stimulations predictable to the subject, as this may skew the results. Rather, stimulations should preferably be made at irregular intervals and/or the subject should be asked for a response when there is actually no stimulation.

The thread used for contacting the mucosa has such properties that the force applied, as determined by the separation between the mucosa and the duct, to a known probability falls within a predetermined range. The thread will act as a force lever, in the sense that the magnitude of the force applied is significantly smaller, as well as confined to a smaller range, that what would be possible to obtain if the force were applied directly using a rigid device.

The magnitude of the stimulation could be measured in a selection of units, such as pressure or force. Given that the contact area between the test stick and the mucosa is at least in principle known but not necessarily homogeneous, the unit selected to describe the stimulation provided by the inventive test sticks is a force unit expressed in milligrams, wherein 1 mN is substantially equivalent to the force of gravity at sea level on a 0.1 g mass. Typical forces provided by the test sticks are in the range from about 0.1 mg to about 500 mg, preferably from about 1 mg to about 100 mg, applied to a typical tip cross section of about 0.001 to about 1.00 mm². For some applications, e.g. when testing very sensitive tissue, even smaller threads may be used. Having read and understood this specification, the skilled person will find suitable dimensions for each application.

The force characteristics may be achieved, for example, using either of the following approaches.

As a first example, threads of different physical properties may be used. The threads may have different diameter, stiffness, material, radius of curvature, etc., or different lengths extending from the elongated portion (duct) of the test stick. A bundle of monofilaments may also be used, wherein for example the number of filaments in a bundle determines the force characteristics. When the tip of the thread is pressed against the mucosa to effect a stimulation, the entire thread is deformed elastically to provide a reactive force.

As a second example, comparatively stiff threads may be used. Rather than using threads of different physical properties, the threads are locally weakened at a point close to the end of the duct such that the thread will bend at the weakened point to a known extent when subjected to a force, thus providing an equal and opposite reaction force. The weakened point may be provided by means of any suitable method, such as by cutting, drilling or by laser ablation. This has already been discussed above in connection with FIG. 5.

For the first example above, using threads of different physical properties, the magnitude of the force applied across the contact surface is affected by several characteristics of the thread, such as the thread diameter, the length of the thread extending from the duct, the curvature of the thread, the stiffness of the thread, and to some extent air humidity and air temperature. Suitable selections will be described in more detail below.

When using the second example above, where the thread has a locally weakened point to provide the desired force, basically the same characteristics as above are relevant. However, in this case it is the characteristics of the weakened point which are of primary interest. For this latter case, an advantage is obtained, as mentioned above, in that the thread will primarily flex in a single plane upon contact, which may facilitate use of the device.

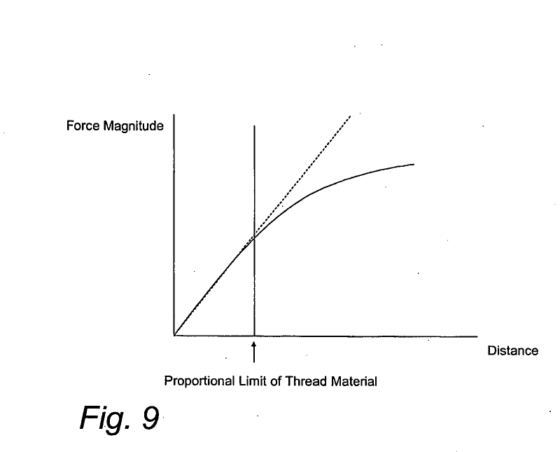

The stimulating force applied to the mucosa by the inventive test stick is produced by the reaction force to the deformation of the thread. After having made contact to the mucosa, the reaction force is initially approximately proportional to the displacement of the thread tip. After some further displacement of the thread tip, the magnitude is no longer proportional to the displacement, but starts to decrease. This is schematically illustrated in FIG. 9.

Hence, the displacement and the modulus of elasticity of the thread combine to provide a probability distribution for the applied force. If the displacement is such that the proportional limit of the thread is exceeded, the probability distribution for the applied force will show a lower probability of large forces, in effect leading to a narrowing of the overall force distribution. Test sticks can be designed to operate both below and above the proportional limit, as long as the material remains elastic and is not permanently deformed. By carefully selecting the modulus of elasticity, a test stick may be designed to have a specific probability distribution for the applied force.

In the following, the parts of the inventive test stick will be described in more detail.

As mentioned above, the test stick 10 may comprise an optional handle 18, allowing the operator to comfortably grip the test stick and apply the tip 16 of the thread 14 against the mucosa or other tissue with sufficient precision. Alternatively, the test stick 10 may be adapted to be mounted in a mechanical manipulator (not shown), and operated indirectly by means of this manipulator, rather than manually by hand.

It is preferred that the elongated portion 12 of the test stick 10 is implemented in the form of a duct or a tube. However, it is also conceivable that the elongated portion is a solid rod or the like. The purpose of the elongated portion, e.g. the duct, is to facilitate for the operator to introduce the stimulating end 16 of the thread 14 into the nasal cavity, for example, to stimulate the nasal mucosa. The duct 12 should be rigid compared to the thread 14, such that the duct will not flex when the thread is subjected to a force, at least not to any material extent. A suitable length for the duct 12 is from about 30 mm to about 100 mm, preferably from about 50 mm to about 70 mm. The duct should also preferably be of comparatively small diameter, in order not to obstruct the view of the operator during the procedure, and to easily pass into the nasal cavity through a nostril. Being a medical instrument, the duct should also be made from a material that will not irritate any human tissue it may contact during the procedure. Preferred materials for the duct are stainless steel or any suitable plastic material.

The thread 14 may be made from surgical suture thread (made of nylon or similar), or other materials having similar physical properties, such as a filament of stainless steel. Preferably, the thread 14 is circular in cross section, having a diameter of about 0.08-1.0 mm, most preferably having a diameter of about 0.40 mm or less. The thread should be reasonable inert and stable, such that its given curvature and stiffness is preserved, within suitable limits, during its shelf-life. Typically, the thread should extend from the duct 2-6 mm. The portion of the thread 14 extending from the duct 12 may be curved, preferably having a radius of curvature of about 1-30 mm, for example about 10 mm or less.

For one exemplary embodiment, the surface area of contact between the thread 14 and the mucosa during testing should not be so large that many nerve endings are stimulated at the same time. If many nerve endings were stimulated, the most sensitive nerve endings would most frequently be triggered, providing a distribution of sensitivity measurements indicating a higher average sensitivity compared to what is representative for the actual, overall sensitivity of the mucosa. It is therefore advised that a sampling procedure is used for measuring the aggregate sensitivity, using a plurality of smaller measurement locations. However, for other embodiments, the contact area may intentionally be made sufficiently large to stimulate a plurality of nerve endings.

The optional handle 18 may be attached to the duct 12, or elongated portion, of the test stick 10 using an articulated joint or a fixed attachment (not shown). An articulated joint is preferred when the handle 18 is not symmetric about the longitudinal axis of the duct 12, in order to facilitate for the operator to stimulate the mucosa from any direction.

The thread 14 extending substantially in an axial direction from the elongated portion 12 may be attached by any suitable means. For example, when the elongated portion has the form of a duct, the thread may be attached thereto by means of an indentation of the duct, a wedge or similar, keeping the thread in place. Alternatively, the thread may be glued to the elongated portion. For further details, reference is made to the earlier description of this specification.

In order to allow proper application of the test stick 10, it should be clearly visible to the operator through the microscope (or other means used for monitoring the procedure). When using an automated, digital system, it may also be desirable that a computer aided system should be able to locate the position of the test stick 10 and/or the geometry of the thread 14 by analysis of a video feed. The test stick 10 is therefore preferably designed for maximum visual clarity, e.g. by having a distinct color for the thread extending from the elongated portion, by having a color marking at the tip of the thread, or by having one or several color markings (such as rings) around the thread at points along the length of the thread extending through the duct. For automated systems, it may be desired to design the thread and the other parts of the test stick such that a camera, CCD or similar is able to capture the entire length of the thread.

It is particularly preferred that the invention is implemented as a test kit, comprising a plurality of test sticks. Such test kit will now be described in more detail.

A test kit 80 comprising five different test sticks 10 is schematically shown in FIG. 8. However, it is understood that the test kit may comprise more or fewer test sticks as needed or desired. For the test kit, normal and abnormal levels of sensitivity in the mucosa should be distinguishable to a high precision, preferably to a probability of more than 0.8 of correct classification across the test subject population. The test kit may be designed to permit the operator to diagnose hyposensitivity, hypersensitivity or both, depending on the requirements of the application at issue. The test kit 80 may further be designed to permit the operator to assess the topological distribution of tactile sensitivity, and to infer the underlying cause of the observed distribution. Moreover, the test kit should preferably provide some means for validation that the test procedure was properly conducted.

Given the above general objectives for the test kit, the number of different test sticks in each kit may be selected as follows.

For a test kit designed to identify hypersensitivity, the kit should contain a test stick with a force confidence interval entirely and immediately below (as measured by threshold force) the normal sensitivity for the test subject population. A positive determination from this test stick would indicate a heightened sensitivity for the subject.

For a test kit designed to identify hyposensitivity, the kit should contain a test stick with a force confidence interval entirely and immediately above (as measured by the threshold force) the normal sensitivity for the test subject population. A negative determination from this test stick would indicate a reduced sensitivity for the subject.

In order to permit the observation of the underlying causes of the topological distribution of tactile sensitivity, the kit may include a test stick having a force magnitude immediately above that of the strongest one used for measuring tactile sensitivity, or otherwise complementing the range of forces applied by the test sticks of the kit.

The test kit should preferably include two test sticks having force characteristics in the extremes of the range of forces that reflect threshold sensory forces typically found in the test subject population. In most cases, this should ensure that at least these two test sticks of the kit give different results (one positive and one negative), indicating that the test sticks have been used in a proper manner during the procedure.

In order to facilitate the measuring procedure, the test sticks should preferably have force characteristics that are sufficiently different such that for most subjects and for most of the time, no two adjacent sticks of the kit give ambiguous readings of the sensitivity. In other words, for any two sticks of the kit having successive force characteristics (in the meaning that they are adjacent in rank with respect to the force delivered), at least one stick should preferably give a clear result (i.e. mainly positive or mainly negative responses during the sampling).

During one example of a sampling procedure, the operator uses different test sticks of the kit in sequence to test the mucosal sensitivity to tactile stimulation. The sequencing of the test sticks within the kit should designed to produce reliable, precise and unbiased results. It is, however, not required for the different test sticks to be applied exactly to the same locations of the mucosa during the samplings of a procedure, since a statistical measure is used for the determination.

Typically, the operator will establish the validity of the measurement procedure by using the two test sticks at the extremes of the force range under consideration; these two sticks may be called control sticks for negative and positive determination. If these two sticks do not produce opposite results, the operator should investigate whether the results are due to mistakes in the operation of the test kit, or whether the subject has indeed a truly extreme tactile sensitivity. The results of the test should not be considered reliable if the control sticks for negative and positive determination do not produce the expected results.

Next, the operator should use different test sticks in the kit, and identify the test stick of lowest force that still produce a positive response from the test subject (i.e. that the subject can sense). During this stage, the sticks may be used in any order.

Once the test stick having the lowest force that can be sensed by the subject has been identified (“the threshold stick”), the consistency and reliability of the measurement may be assessed by using the sticks immediately above and below in force (if applicable), regardless of whether these have been tested before, and then using the threshold stick again. The procedure may be repeated a number of times. This should produce a negative and a positive control measurement for identifying the threshold stick.

As noted above, each test stick can be designed for a specific probability function of applied force by carefully selecting its modulus of elasticity or other suitable property. The test kit is then composed of a number of test sticks of different probability functions for the applied force. For example, the force confidence intervals for the respective test sticks may be distributed logarithmically over the range of contact forces.

The force applied by the test sticks will now be discussed in some further detail. Generally, for small displacements of the thread, the reaction force (and thus the force applied to the mucosa) will be substantially proportional to the displacement. For larger displacements, however, the force applied force will deviate from this proportionality. This is schematically illustrated in FIG. 9. During use of the test stick, the force applied to the mucosa will to some extent depend upon how much displacement, or deflection, is brought to the thread. The magnitude of the force can thus be described by a force confidence interval, or force probability distribution function.

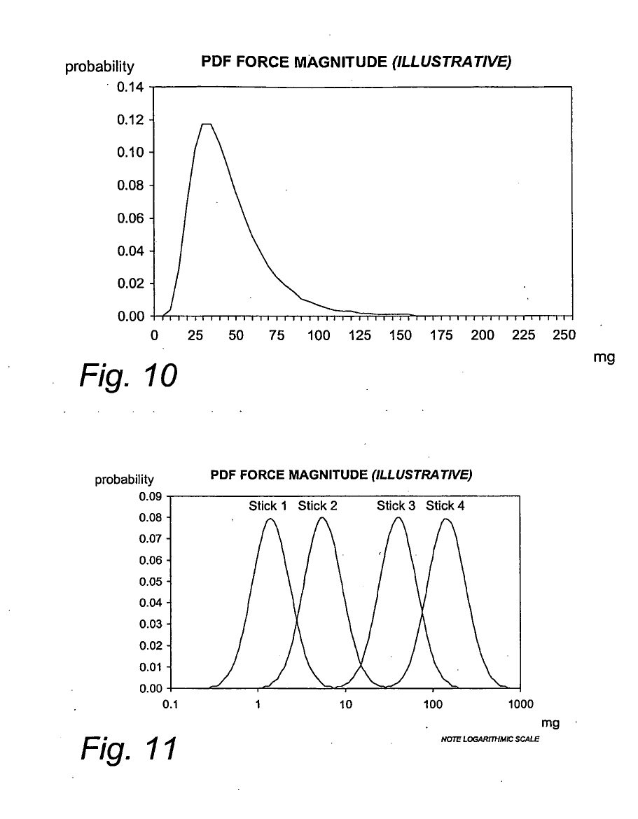

For example, a test stick (its thread) may be designed to have the largest probability of delivering a stimulation force of about 30 mg (see FIG. 10). For such a stick, it will be most probable that a force of about 30 mg is applied during use. However, other magnitudes of force may be applied depending upon how the stick is brought into contact with the mucosa each time. The ability to obtain a measure corresponding to reactive forces with known statistical properties from the stick is one of the reasons for suggesting that a sampling procedure is employed when using the inventive device and kit. A sampling procedure is also preferred since nerve sensitivity is not uniformly distributed across the mucosa, and such sampling procedure ensures that the measurement procedure provides a more representative measure for the area of mucosa under investigation.

FIG. 11 shows an illustration of the similar kind, but for a test kit comprising four test sticks. In order to have different force applied for each different test stick, the probability functions for the applied force are distributed among the test sticks over the force range of interest. Note that the abscissa in the graph of FIG. 11 is logarithmic. Hence, for the test kit corresponding to the distributions illustrated in FIG. 11, the sticks have highest probability of applying a force of about 1 mg, about 5 mg, about 50 mg and about 100 mg, respectively. It should be noted, however, that this is merely an example, and many other selections and distributions can be made.

### CONCLUSION

A device for testing tactile sensitivity of tissue, such as mucosa, has been presented. The device is specifically designed to be used in difficult to reach parts of a test subject, e.g. in the nasal cavity or in the throat (for reaching the larynx). The inventive test device relies upon a principle where a thread (monofilament or bundle of filaments) extends substantially axially from an elongated portion, wherein the thread has been given a curvature or a bend in order to provide a lateral point of contact away from the longitudinal axis of the elongated portion of the device. It is also proposed to have a test kit, comprising a plurality of test devices, wherein the devices of the kit are used during testing for different tactile sensitivity thresholds. The stimulating force delivered from each test device need not be strictly known, but a probability distribution of the applied force may be used.

Hence, there is disclosed a device for testing tactile sensitivity of tissue, such as nasal mucosa, comprising an elongated portion to which there is attached a thread. The thread is adapted to be brought into contact with the tissue to be tested, wherein the thread is curved or otherwise bent so as to define a lateral contact point away from the axis of the elongated portion. Preferably, the thread is axially attached to the elongated portion, and exhibits a curvature or bent to define a lateral contact point about 10 mm or less away from the axis of the elongated portion.

1. A device for testing tactile sensitivity of nasal mucosa, comprising: an elongated portion for insertion into the nasal cavity or other hard to reach location of a patient; and a thread extending from one end of said elongated portion; wherein the thread is curved or otherwise bent so as to define a lateral contact point to be brought into contact with tissue to be tested for tactile sensitivity.

2. The device of claim 1, wherein the thread extends axially from the elongated portion.

3. The device of claim 1, wherein the length and curvature of the thread is such as to define a lateral contact point no more than about 10 mm, preferably no more than about 5 mm, off the axis of the elongated portion.

4. The device of claim 1, wherein the elongated portion comprises a duct, in which the axially extending thread is arranged.

5. The device of claim 1, wherein the thread is designed to provide a contact force in the range from about 0.1 mg to about 500 mg upon contact, preferably from about 1 mg to about 100 mg.

6. The device of claim 5, wherein the contact force is determined by physical properties of the thread, such as diameter, stiffness, material, curvature, uniformity or homogeneity.

7. The device of claim 5, wherein the contact force is determined by way of a locally weakened point on the thread to provide a bending knee.

8. The device of claim 1, wherein the thread has a tip adapted for contact, said tip having a cross section from about 0.001 mm² to about 1.00 mm².

9. The device of claim 1, further comprising a handle attached to the elongated portion, for facilitating handheld operation of the device.

10. The device of claim 9, wherein the handle is angled from the longitudinal axis of the elongated portion, and wherein the handle is attached to said portion by way of an articulated joint permitting rotation of the handle with respect to said portion.

11. The device of claim 1, further comprising an attachment for mounting the device in a mechanical manipulator.

12. The device of claim 1, wherein the thread is made from nylon or stainless steel, and preferably from surgical suture thread.

13. The device of claim 1, wherein the thread extends 2-6 mm from the free end of the elongated portion.

14. The device of claim 1, wherein the extending length of the thread has a radius of curvature of about 1-10 mm.

15. A test kit for testing tactile sensitivity of nasal mucosa, comprising a plurality of test devices according to claim 1; wherein each device of the kit is adapted to apply a different force to tissue to be tested for tactile sensitivity.

16. The test kit of claim 15, comprising at least three test devices, preferably at least five test devices.

17. The test kit of claim 15, comprising a test device having a force confidence interval corresponding to a value below a normal sensitivity for a test subject population, the test kit thereby being adapted to identify hypersensitivity.

18. The test kit of claim 15, comprising a test device having a force confidence interval corresponding to a value above a normal sensitivity for a test subject population, the test kit thereby being adapted to identify hyposensitivity.

19. The test kit of claim 15, comprising one positive test device designed to provide primarily positive test results, and one negative test device designed to provide primarily negative test results, said positive and negative test devices thus providing an indication that the test kit is being used in a proper manner.
